(19) United States
(12) Patent Application Publication
(10) Pub. No.: US 2013/0260442 A1
(43) Pub. Date: Oct. 3, 2013

(54) CARBON DIOXIDE CAPTURE PROCESS WITH CATALYTICALLY-ENHANCED SOLVENT AND PHASE SEPARATION

(71) Applicant: Frederic Vitse, Knoxville, TN (US)

(72) Inventor: Frederic Vitse, Knoxville, TN (US)

(73) Assignee: ALSTOM TECHNOLOGY LTD, Baden (CH)

(21) Appl. No.: 13/433,694

(22) Filed: Mar. 29, 2012

(51) Int. Cl. B01D 53/62; C12M 1/12

(52) U.S. Cl. 435/266; 435/283.1

(57) ABSTRACT

A system for the regeneration of carbon dioxide from a flue gas comprises an absorption vessel configured to receive a catalytically-enhanced solvent stream and a flue gas stream having CO₂, the catalytically-enhanced solvent stream and the flue gas stream being contacted in the absorption vessel to provide a catalytically-enhanced CO₂-rich outlet stream; a crystallizer configured to receive the catalytically-enhanced CO₂-rich outlet stream from the absorption vessel and produce a two-phase outlet solvent stream having crystals; a separator configured to receive the outlet solvent stream having crystals and separate the outlet solvent stream having crystals into a catalyst solvent stream and a CO₂-rich non-catalyst solvent stream; and a regenerator vessel configured to receive the CO₂-rich non-catalyst solvent stream from the separator and produce a CO₂ stream and a regenerator outlet solvent stream, the regenerator outlet solvent stream combined with catalyst for return to the absorption vessel as the catalytically-enhanced solvent stream.

### TECHNICAL FIELD

The present disclosure generally relates to a system for the capture of carbon dioxide (CO₂) with a solvent and the regeneration of the CO₂ from the solvent. The present disclosure more particularly relates to a system that absorbs CO₂ into a solvent that is enhanced with a catalyst, the catalyst being subsequently separated from the CO₂ using phase separation, and the regeneration of the CO₂ from catalyst-free solvent.

### BACKGROUND

The combustion of carbon- and hydrogen-containing fuel such as oil, coal, and natural gas generally results in the production of a flue gas stream containing contaminant emissions in the form of particulates, hydrocarbons, SOₓ, NOₓ, and the like. One particular hydrocarbon contaminant is CO₂, which is typically referred to as a “greenhouse gas.” Although CO₂ is considered an atmospheric contaminant, it has various beneficial uses, and so it is often absorbed from a flue gas into a solvent, regenerated from the solvent, and captured.

Regenerating the CO₂ from the flue gas is typically carried out by contacting the flue gas with the solvent, stripping away the solvent, and isolating and compressing the CO₂. Steam is often used to strip the solvent. The use of steam to strip the solvent, however, generally involves high temperatures and pressures, which reduce the energy requirements for compression of the CO₂ but which detrimentally affect the stability of the solvent. Conversely, regeneration of the CO₂ at relatively low temperature and pressure does not detrimentally affect the solvent, but it does increase the energy needed for compression of the CO₂. Thus, the efficient capture of CO₂ by regeneration processes that involve absorbing the CO₂ into solvent and stripping the solvent involve balancing the energy requirements for the actual regeneration of the CO₂ from the solvent against the energy requirements for the compression of the CO₂.

### SUMMARY

According to one aspect disclosed herein, a system for the regeneration of carbon dioxide from a flue gas comprises an absorption vessel configured to receive a catalytically-enhanced solvent stream and a flue gas stream having CO₂, the catalytically-enhanced solvent stream and the flue gas stream being contacted in the absorption vessel to provide a catalytically-enhanced CO₂-rich outlet stream. The system also comprises a crystallizer configured to receive the catalytically-enhanced CO₂-rich outlet stream from the absorption vessel and produce a two-phase outlet solvent stream having crystals. A separator is configured to receive the outlet solvent stream having crystals and separate the outlet solvent stream having crystals into a catalyst solvent stream and a CO₂-rich non-catalyst solvent stream. The system further includes a regenerator vessel configured to receive the CO₂-rich non-catalyst solvent stream from the separator and produce a CO₂ stream and a regenerator outlet solvent stream, the regenerator outlet solvent stream being combined with catalyst for return to the absorption vessel as the catalytically-enhanced solvent stream.

According to another aspect disclosed herein, a system for the capture, regeneration, and sequestration of CO₂ comprises a packed column comprising a vessel and a packing material therein, the packed column configured to receive a catalytically-enhanced amine-based solvent stream and a flue gas stream having CO₂, the catalytically-enhanced amine-based solvent stream and the flue gas stream being contacted in the packed column in a counter-current arrangement to provide a catalytically-enhanced CO₂-rich outlet stream. The system further includes a crystallizer configured to receive the catalytically-enhanced CO₂-rich outlet stream from the absorption vessel and produce a two-phase outlet solvent stream having crystals. A cyclone separator is configured to receive the outlet solvent stream having crystals and separate the outlet solvent stream having crystals into a catalyst solvent stream and a CO₂-rich non-catalyst solvent stream. A stripping column receives the CO₂-rich non-catalyst solvent stream from the cyclone separator and produces a CO₂ stream and an outlet solvent stream, the outlet solvent stream being combined with catalyst for return to the packed column as the catalytically-enhanced amine-based solvent stream. A sequestration apparatus is in communication with the stripping column and is configured to receive the CO₂ stream therefrom.

According to another aspect disclosed herein, a method for removing CO₂ from a flue gas comprises contacting a catalytically-enhanced amine-based solvent stream and a flue gas stream having CO₂ in an absorption vessel, the catalytically-enhanced amine-based solvent stream and the flue gas stream being contacted in the absorption vessel in a counter-current arrangement; directing a catalytically-enhanced CO₂-rich outlet stream from the absorption vessel to a crystallizer; forming the catalytically-enhanced CO₂-rich outlet stream in the crystallizer into a first phase having catalyst and solvent and a second phase having solvent and CO₂; directing the first phase back to the absorption vessel; directing the second phase to a stripping column; stripping the second phase having solvent and CO₂ to separate the solvent from the CO₂; sequestering the CO₂ from the second phase; and returning the solvent from the second phase back to the absorption vessel.

The above described and other features are exemplified by the following Figures and detailed description.

### BRIEF DESCRIPTION OF THE DRAWINGS

Referring now to the Figures, which are exemplary embodiments, and wherein the like elements are numbered alike:

FIG. 1 is a schematic representation of a flue gas processing system for the capture of CO₂; and

FIG. 2 is a schematic representation of an absorption loop and a regeneration loop of the flue gas processing system of FIG. 1.

### DETAILED DESCRIPTION

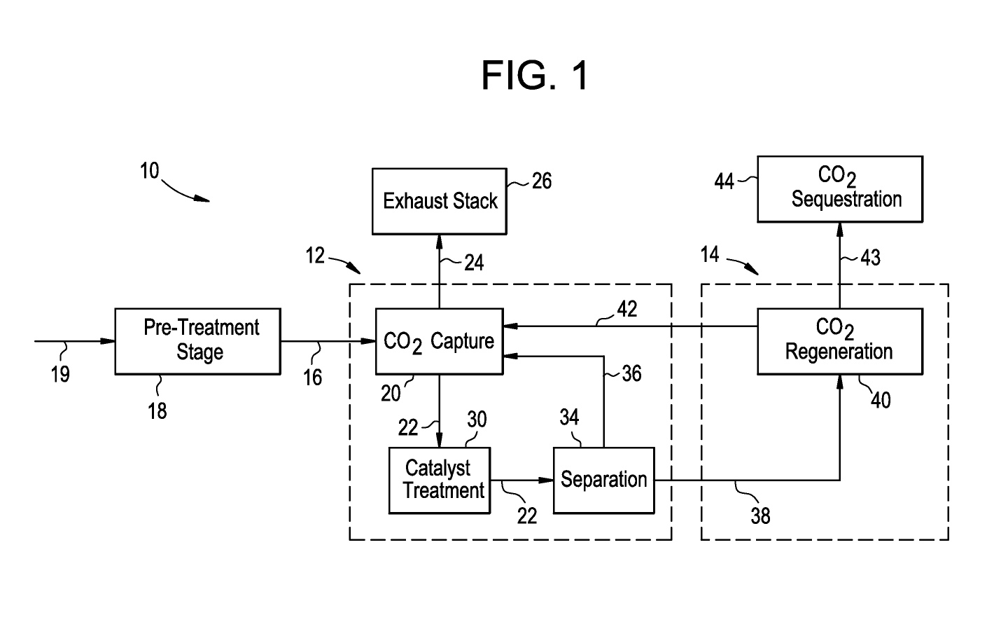

As shown in FIG. 1, a system for the capture of CO₂ from a flue gas using an amine-based solvent and phase separation is designated generally by the reference number 10 and is hereinafter referred to as “system 10.” The flue gas is a product of a combustion process occurring in a boiler, furnace, or the like. System 10 is defined by an absorption loop 12 and a regeneration loop 14. In the absorption loop 12, the CO₂ in the flue gas is captured by being absorbed in the solvent, which is enhanced with a catalyst to facilitate absorption of the CO₂. Once the CO₂ is absorbed into the solvent, a first liquid phase containing catalyst and solvent is separated from a second liquid phase containing CO₂ and solvent. The first liquid phase containing catalyst and solvent is maintained as a stream in the absorption loop 12. The second liquid phase containing CO₂ and the solvent (with substantially no catalyst) is directed to the regeneration loop 14 where the CO₂ is separated from the solvent and sequestered. The solvent is then returned to the absorption loop 12.

The absorption loop 12 of the system 10 receives the flue gas as a flue gas stream 16 from a flue gas pre-processing stage 18, which receives a process stream 19 from the combustion process. The flue gas pre-processing stage 18 may include one or more devices such as, but not limited to, a scrubber, a dust removal system, a pre-heater, or the like. From the flue gas pre-processing stage 18, the flue gas stream 16 is directed to a CO₂ capture system 20 and contacted with catalytically-enhanced solvent to produce a CO₂-rich outlet stream 22 and a treated flue gas stream 24. The treated flue gas stream 24 is directed to an exhaust stack 26, and the CO₂-rich outlet stream 22 is directed to a catalyst treatment system 30. From the catalyst treatment system 30, the CO₂-rich outlet stream 22 is directed to a separation apparatus 34 that separates the CO₂-rich outlet stream 22 into an absorber inlet stream 36 that is semi-lean in CO₂ and contains catalyst (the first liquid phase or solid/liquid phase) and a regeneration stream 38 that is rich in CO₂ and contains substantially no catalyst (the second liquid phase or solid/liquid phase). The absorber inlet stream 36 is directed back to the CO₂ capture system 20.

The regeneration stream 38 is directed to the regeneration loop 14 and, in particular, to a CO₂ regeneration apparatus 40. In the CO₂ regeneration apparatus 40, the CO₂ is separated from the regeneration stream 38, taken off as a CO₂ gas stream 43, and sequestered in a sequestration apparatus 44. A regeneration apparatus takeoff stream 42 that is solvent lean in CO₂ is directed back to the absorption loop 12.

The catalytically-enhanced solvent employed in system 10 is a high cyclic capacity amine-based solvent or ammonia. The solvent may be, for example, a tertiary amine (e.g., triethylamine, trimethylamine, or the like) or an amino acid. The catalyst used to enhance the solvent may be an enzyme. The enzyme may be a metalloenzyme such as, for example, carbonic anhydrase. Because the conditions at which the CO₂ capture process 20 operates is conducive to the catalyst, and because the catalyst is maintained within the absorption loop 12 (and is substantially prevented from entering the regeneration loop 14 due to the separation apparatus 34), the kinetics of the solvent are enhanced by the catalyst, thereby allowing for advantageous separation and sequestration of the CO₂ in the regeneration loop 14.

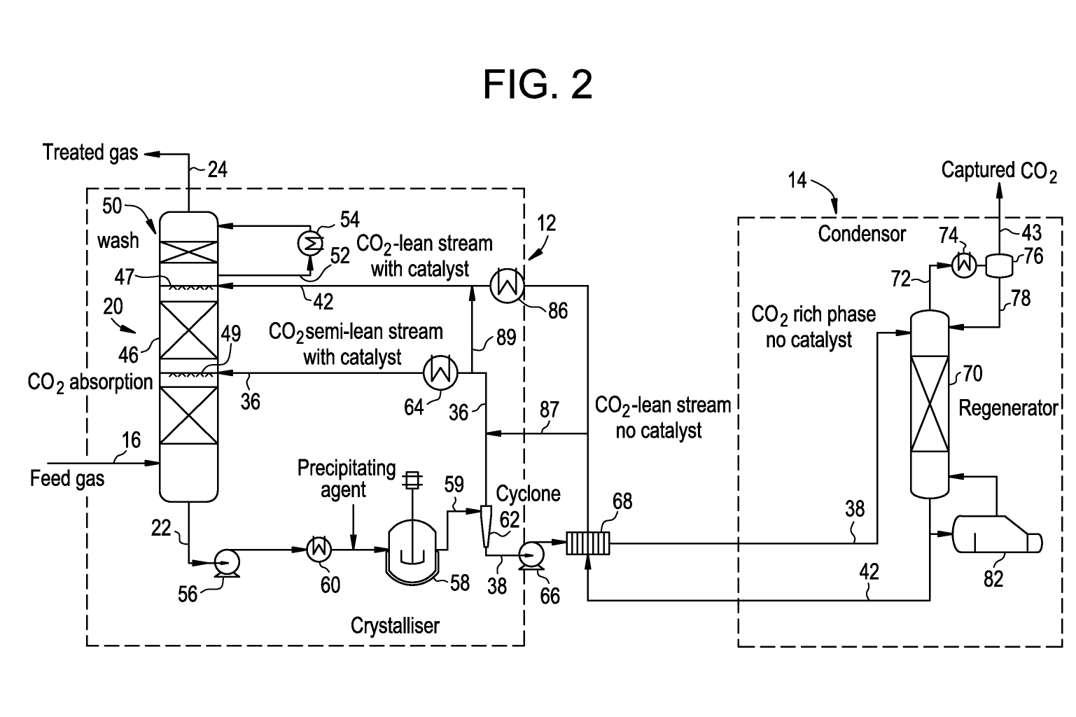

As shown in FIG. 2, the CO₂ capture system 20 of the absorption loop 12 comprises an absorber vessel 46 configured to apply the solvent to the flue gas stream 16 from the flue gas pre-processing stage 18. The absorber vessel 46 is a packed column containing packing material either arranged in a structured configuration or randomly dumped within the vessel. The regeneration apparatus takeoff stream 42 is received into the absorber vessel 46 and distributed across the upper portion thereof via a first liquid distribution system 47. The first liquid distribution system 47 distributes the solvent of the regeneration apparatus takeoff stream 42 substantially uniformly over the cross-sectional geometry of the packed column, thereby allowing the solvent to permeate the packing material and flow downward in a substantially even manner. The absorber inlet stream 36 from the separation apparatus 34 is also received into the absorber vessel 46 at a point below the inlet for the regeneration apparatus takeoff stream 42 and is distributed across the absorber vessel via a second liquid distribution system 49. The flue gas stream 16 is introduced to the absorber vessel 46 at or near the bottom of the packed column. The flow of solvent downward through the packing material and the upward flow of the flue gas allow for the counter-current contact of the solvent (lean to semi-lean in CO₂) and the flue gas, thereby allowing the solvent to become enriched in CO₂ as it flows to the bottom of the absorber vessel 46.

The absorber vessel 46 includes a wash section 50 located at the upper portion thereof. The wash section 50 circulates water through the upper portion of the absorber vessel 46 by taking a water stream 52 off the packed column at a point above the point at which the regeneration apparatus takeoff stream 42 enters the absorber vessel 46 and directing the water stream 52 to the top of the absorber vessel 46. The water stream 52 is cooled via a wash chiller 54. Use of the wash section 50 allows the cooled water to remove any solvent that may be present in the flue gas prior to the flue gas being ejected as the treated flue gas stream 24 directed to the exhaust stack 26.

The CO₂-rich outlet stream 22 from the bottom of the absorber vessel 46 is fed to a pump 56, which pumps the CO₂-rich outlet stream 22 to a crystallizer 58. As the CO₂-rich outlet stream 22 is pumped to the crystallizer 58, the stream is cooled via an absorber bottoms chiller 60. Prior to the CO₂-rich outlet stream 22 entering the crystallizer 58, precipitating agent 62 such as, for example, potassium carbonate, may be added (optional) to facilitate the formation of crystals. The precipitating agent 62 is any suitable agent capable of causing the catalyst to precipitate from the solvent or otherwise forming the first liquid phase containing the catalyst relative to the second liquid phase such that the first liquid phase is non-miscible in the second liquid phase.

From the crystallizer 58, a binary liquid phase stream 59 (two-phase outlet solvent stream having crystals) is directed to the separation apparatus 34, which may be, for example, a cyclone separator 62. In the cyclone separator 62, the binary liquid phase stream 59 is spun down to separate the stream into a light phase containing the first liquid phase or solid/liquid phase (catalyst, solvent, and a small amount of CO₂) and a heavy phase containing the second liquid phase or solid/liquid phase (solvent and CO₂). The presently disclosed system 10, however, is not limited to the separation of the two phases using only the cyclone separator 62. In particular, the separation of the first liquid phase from the second liquid phase may be due at least in part to the cooling of the solvent into a thermodynamic region where the solubility limit of the CO₂ in the solvent is exceeded, thereby allowing the second liquid phase to separate from the first liquid phase.

The first liquid phase is taken off the cyclone separator 62 as the CO₂ semi-lean absorber inlet stream 36, and the second liquid phase is taken off the cyclone separator as a heavy phase containing the regeneration stream 38 rich in CO₂ and having substantially no catalyst. The absorber inlet stream 36 is directed through a chiller 64 and back to the absorber vessel 46. The regeneration stream 38 is pumped (via a pump 66) through a heat exchanger 68 and from the absorption loop 12 to the regeneration loop 14.

The regeneration stream 38 leaving the heat exchanger 68, which is rich in CO₂ and contains substantially no catalyst, is directed to the upper portion of a regenerator vessel or stripping column 70. The stripping column 70 is a cylindrically-shaped container having gas-liquid contacting devices for facilitating mass transfer. Such gas-liquid contacting devices include, but are not limited to, random packing material, structured packing material, and trays. When the gas-liquid contacting devices include random or structured packing material, a liquid distribution system (not shown) may be employed to distribute the regeneration stream 38 substantially uniformly over the cross-sectional geometry of the stripping column 70, thereby allowing the solvent to permeate the packing material and flow downward in a substantially even manner.

During operation, the stripping column 70 is heated to cause the solvent of the regeneration stream 38 to vaporize with the CO₂. An overhead stream 72 containing solvent vapor and CO₂ is taken off the top of the stripping column 70 and directed to a condenser 74, which at least partially condenses the solvent and leaves the CO₂ as a gas. The at least partially condensed solvent is directed to a reflux drum 76. From the reflux drum 76, the CO₂ gas stream 43 is removed and directed to the sequestration apparatus 44, and a stream of condensed solvent (which may contain some CO₂) is returned to the top of the stripping column 70.

The stripping column 70 is heated via a reboiler 82, which receives at least a portion of the regeneration apparatus takeoff stream 42 from the bottom of the stripping column, heats the portion received, and returns it to the stripping column.

The regeneration apparatus takeoff stream 42 taken from the bottom of the stripping column 70 and not directed to the reboiler 82 is directed to the heat exchanger 68. The regeneration apparatus takeoff stream 42, which is substantially at the operating temperature of the stripping column 70, transfers heat to the regeneration stream 38 in the heat exchanger 68. The heat exchanger 68 may be of the plate and frame type, or it may be of the shell and tube type.

From the heat exchanger 68, the regeneration apparatus takeoff stream 42, which is lean in CO₂ and contains substantially no catalyst, is received into the upper portion of the absorber vessel 46 (as described above). The regeneration apparatus takeoff stream 42 is cooled using an inlet chiller 86. To control the temperature of the streams into the absorber vessel 46, however, at least a portion of the regeneration apparatus takeoff stream 42 may be directed around the chiller 86 via one or more of line 87 and line 89. While not shown, valves positioned in line 87 and line 89 allow for the adjustable control of the flow through line 87 and 89, thereby allowing for the adjustable control of the CO₂ semi-lean absorber inlet stream 36 and the regeneration apparatus takeoff stream 42 into the absorber vessel 46. In doing so, the volumetric flow rate of the regeneration apparatus takeoff stream 42 through the chiller 86 can be varied, thereby allowing for the proportion of cooling to the streams fed to the absorber vessel 46 (the CO₂ semi-lean absorber inlet stream 36 and the regeneration apparatus takeoff stream 42) to be varied.

While the invention has been shown and described with respect to the detailed embodiments thereof, it will be understood by those of skill in the art that various changes may be made and equivalents may be substituted for elements thereof without departing from the scope of the invention. In addition, modifications may be made to adapt a particular situation or material to the teachings of the invention without departing from the essential scope thereof. Therefore, it is intended that the invention not be limited to the particular embodiments disclosed in the above detailed description, but that the invention will include all embodiments falling within the scope of the foregoing description.

What is claimed is:

1. A system for the regeneration of carbon dioxide from a flue gas, the system comprising:
A CO₂ capture system configured to receive a catalytically-enhanced solvent stream and a flue gas stream having CO₂, the catalytically-enhanced solvent stream and the flue gas stream being contacted in the CO₂ capture system to provide a catalytically-enhanced CO₂-rich outlet stream;
a catalyst treatment system configured to receive the catalytically-enhanced CO₂-rich outlet stream from the CO₂ capture system and produce a two-phase outlet solvent stream having crystals;
a separation apparatus configured to receive the outlet solvent stream having crystals and separate the outlet solvent stream having crystals into a catalyst solvent stream and a CO₂-rich non-catalyst solvent stream; and
a CO₂ regeneration apparatus configured to receive the CO₂-rich non-catalyst solvent stream from the separation apparatus and produce a CO₂ stream and a regenerator outlet solvent stream, the regenerator outlet solvent stream being combined with catalyst for return to the CO₂ capture system as the catalytically-enhanced solvent stream.

2. The system of claim 1, wherein the catalytically-enhanced solvent stream received into the CO₂ capture system comprises a CO₂-lean stream having catalyst and a CO₂-semi-lean stream having catalyst.

3. The system of claim 1, wherein the separation apparatus is a cyclone separator.

4. The system of claim 1, further comprising a heat exchanger positioned between the CO₂ regeneration apparatus and the separation apparatus and configured to exchange heat between the regenerator outlet solvent stream and the CO₂-rich non-catalyst solvent stream from the separation apparatus.

5. The system of claim 1, wherein the CO₂ regeneration apparatus is a stripping column.

6. The system of claim 5, further comprising a reboiler in fluid communication with the stripping column.

7. The system of claim 1, further comprising a sequestration apparatus in communication with the CO₂ regeneration apparatus and configured to receive the CO₂ stream therefrom.

8. The system of claim 1, wherein the catalytically-enhanced solvent stream comprises an amine-based solvent selected from the group consisting of tertiary amines and amino acids.

9. The system of claim 1, wherein a catalyst in the catalytically-enhanced solvent stream comprises an enzyme.

10. A system for the capture, regeneration, and sequestration of CO₂, the system comprising:
a packed column comprising a vessel and a packing material therein, the packed column configured to receive a catalytically-enhanced amine-based solvent stream and a flue gas stream having CO₂, the catalytically-enhanced amine-based solvent stream and the flue gas stream being contacted in the packed column in a counter-current arrangement to provide a catalytically-enhanced CO₂-rich outlet stream;
a crystallizer configured to receive the catalytically-enhanced CO₂-rich outlet stream from the absorption vessel and produce a two-phase outlet solvent stream having crystals;
a cyclone separator configured to receive the outlet solvent stream having crystals and separate the outlet solvent stream having crystals into a catalyst solvent stream and a CO₂-rich non-catalyst solvent stream;
a stripping column configured to receive the CO₂-rich non-catalyst solvent stream from the cyclone separator and produce a CO₂ stream and an outlet solvent stream, the outlet solvent stream being combined with catalyst for return to the packed column as the catalytically-enhanced amine-based solvent stream; and
a sequestration apparatus in communication with the stripping column and configured to receive the CO₂ stream therefrom.

11. The system of claim 10, further comprising a heat exchanger positioned between the cyclone separator and the stripping column and configured to exchange heat between the outlet solvent stream and the CO₂-rich non-catalyst solvent stream from the cyclone separator.

12. The system of claim 10, further comprising a reboiler in communication with the stripping column.

13. The system of claim 10, further comprising a reflux drum in fluid communication with the stripping column.

14. The system of claim 10, wherein the catalytically-enhanced amine-based solvent stream comprises at least one of a tertiary amine and an amino acid.

15. The system of claim 10, wherein a catalyst in the catalytically-enhanced amine-based solvent stream comprises an enzyme.

16. A method for removing CO₂ from a flue gas, the method comprising:
contacting a catalytically-enhanced amine-based solvent stream and a flue gas stream having CO₂ in an absorption vessel, the catalytically-enhanced amine-based solvent stream and the flue gas stream being contacted in the absorption vessel in a counter-current arrangement;
directing a catalytically-enhanced CO₂-rich outlet stream from the absorption vessel to a crystallizer;
forming the catalytically-enhanced CO₂-rich outlet stream in the crystallizer into a first phase having catalyst and solvent and a second phase having solvent and CO₂;
directing the first phase back to the absorption vessel;
directing the second phase to a stripping column;
stripping the second phase having solvent and CO₂ to separate the solvent from the CO₂;
sequestering the CO₂ from the second phase; and
returning the solvent from the second phase back to the absorption vessel.

17. The method of claim 16, further comprising transferring heat from the solvent being returned from the stripping column to the absorption vessel to the second phase directed to the stripping column.

18. The method of claim 16, further comprising water-washing the solvent in the absorption vessel.

19. The method of claim 16, wherein the step of forming the catalytically-enhanced CO₂-rich outlet stream in the crystallizer into a first phase having catalyst and solvent comprises precipitating the catalyst using potassium carbonate.

20. The method of claim 16, wherein the step of forming the catalytically-enhanced CO₂-rich outlet stream in the crystallizer into a first phase having catalyst and solvent and a second phase having solvent and CO₂ comprises cooling the amine-based solvent into a thermodynamic region such that the solubility limit of the CO₂ in the amine-based solvent is exceeded.
